(19) United States
(12) Patent Application Publication
(10) Pub. No.: US 2002/0147461 A1
(43) Pub. Date: Oct. 10, 2002

(54) Apparatus and methods for closing openings in spinal discs

(76) Inventors: William N. Aldrich, Napa, CA (US); Michael T. Carley, San Jose, CA (US); Stephen M. Salmon, Napa, CA (US); Richard S. Ginn, San Jose, CA (US); W. Martin Belef, San Jose, CA (US)

Correspondence Address: LYON & LYON LLP, 633 WEST FIFTH STREET, SUITE 4700, LOS ANGELES, CA, 90071, US

(21) Appl. No.: 09/828,030

(22) Filed: Apr. 6, 2001

(51) Int. Cl. A61B 17/08

(52) U.S. Cl. 606/213

(57) ABSTRACT

A closure device includes a body defining a longitudinal axis, and a plurality of tissue engaging elements that are expandable between contracted and expanded conditions, and preferably biased to extend outwardly from the body in the expanded condition. The closure device may include a generally planar body including tines extending from its outer edge, an elongate body including annular flanges extending from its peripheral surface, or an annular body including tissue engaging elements extending from its proximal portion. An opening is created through the annulus fibrosis into a disc, and a procedure is performed within the disc. The closure device is introduced into the opening, and the tissue engaging elements are expanded to engage tissue surrounding the opening, thereby anchoring the closure device within the opening. Alternatively, the closure device may be a threaded plug that is threaded into the opening after completing the procedure to seal the opening.

### FIELD OF THE INVENTION

The present invention relates generally to apparatus and methods for closing openings through tissue, and more particularly to devices and methods for closing an opening in a spinal disc created during a diagnostic and/or therapeutic procedure.

### BACKGROUND

Various apparatus and methods have been suggested for treating spinal discs when they degenerate or otherwise become injured. For example, spinal fixation, i.e., fixing the vertebrae on either side of an injured disc relative to one another, is a commonly used treatment. This may involve inserting pedicle screws or other anchors into the vertebrae, and securing rods, wires, and the like between the vertebrae, thereby substantially removing much of the forces acting on the disc during subsequent activity by the patient. Such fixation devices, however, may substantially impair free movement by the patient, because relative movement of the vertebrae is intentionally fixed. As an alternative to fixation, an injured disc may be completely removed and replaced with a prosthesis.

U.S. Pat. Nos. 5,549,679 and 5,571,189, issued to Kuslich, disclose implanting a porous bag into a spinal disc to promote fusion of the adjacent vertebrae. A bore is formed through the annulus fibrosis to gain access to the interior of the annulus. A hollow space is formed within the interior of the annulus that exposes surface areas of the vertebrae on either side of the disc. A porous bag is inserted into the space and filled with finely chopped cancelous bone chips. The bag is formed from a porous fabric or a polymeric material having a plurality of perforations formed therein to promote bone ingrowth into the space and ensure that fusion occurs.

Once the bag is filled to a desired pressure, the inlet of the bag is sealed using a threaded cap, a purse-string closure, a staple, or tying a knot in the bag. A patch is then attached to the exterior of the annulus fibrosis in an attempt to seal the entry passage used to access the interior of the disc. Because of the significant stresses experienced by spinal discs during normal physical activity, however, such patches may not resist the substantial pressure experienced within a spinal disc during normal physical activity, and therefore may not effectively seal the opening in the annulus fibrosis.

U.S. Pat. No. 6,022,376, issued to Assell et al., discloses implanting one or more capsule-shaped prosthetic implants within a spinal disc. The implants are formed from a polymer jacket containing a polymer core, such as hydrogel, that is in a flowable state. A flap is cut into the annulus fibrosis for each implant, nucleus pulposus material is removed from the disc to create a space, the jacket is inserted into the space, and polymer core material is introduced to fill the jacket. Alternatively, the jacket, already filled with the polymer core, may be implanted within the disc space. After the implant is implanted, the flap is sewn closed. Merely sewing the flap in the annulus fibrosis closed, however, may not effectively seal the opening in the annulus fibrosis.

Accordingly, apparatus and methods for sealing or closing openings in spinal discs would be considered useful.

### SUMMARY OF THE INVENTION

The present invention relates generally to apparatus and methods for closing openings through tissue, and more particularly to devices and methods for closing an opening in an intervertebral disc created during a diagnostic and/or therapeutic procedure.

According to one aspect of the present invention, a device for closing an opening in a spinal disc is provided that generally includes a body defining a longitudinal axis and having a size for insertion into an opening in a disc, and a plurality of tissue engaging elements on the body. The tissue engaging elements are expandable from a contracted condition towards an expanded condition, the tissue engaging elements extending outwardly from the body substantially transversely to the longitudinal axis in the expanded condition. Preferably, the tissue engaging elements are biased towards the expanded condition, but may be resiliently deflected into the contracted condition. In the contracted condition, the tissue engaging elements may define a more acute angle with respect to the longitudinal axis than in the expanded condition. For example, in some embodiments, at least a portion of the tissue engaging elements may extend substantially parallel to the longitudinal axis in the contracted condition.

In one embodiment, the body may include a generally planar body including an outer edge. The longitudinal axis may extend substantially normal to a surface of the planar body, e.g., at a point of symmetry. In a relaxed state free from external forces, the planar body may be substantially flat or may define a slightly concave shape. The tissue engaging elements may extend from the outer edge of the planar body substantially tangentially from the planar body in the expanded condition. The tissue engaging elements may be deflected towards an axial orientation substantially parallel to the longitudinal axis in the contracted condition. The planar body may be deflectable to assume a concave shape when the tissue engaging elements are deflected towards the contracted condition or may remain substantially planar as the tissue engaging elements assume the contracted condition.

In another embodiment, the body may be an elongate body defining a peripheral surface extending between proximal and distal ends of the elongate body. The tissue engaging elements may extend along the peripheral surface substantially perpendicular to the longitudinal axis. The outer tips of the tissue engaging elements being deflectable towards the elongate body to define the contracted condition. For example, the tissue engaging elements may be a plurality of annular flanges extending from the peripheral surface, with the flanges disposed adjacent one another along the longitudinal axis. Preferably, the outer tip of each flange is biased towards the expanded condition such that the tip extends substantially transversely from the peripheral surface. More preferably, in a relaxed state, the tissue engaging elements define an acute angle with the peripheral surface along the longitudinal axis. Alternatively, the flanges may define a helical thread pattern extending along the peripheral surface.

In yet another embodiment, the body may be a generally annular-shaped member, with the longitudinal axis extending between proximal and distal portions thereof. The plurality of tissue engaging elements may extend from the proximal portion, and are preferably biased from the contracted condition towards the expanded condition. A plurality of leaflets extend from the distal portion, the leaflets being biased from an open condition towards a closed condition. In the open condition, the leaflets may be oriented distally substantially parallel to the longitudinal axis, while in the closed condition, the leaflets may be oriented towards one another for substantially closing an opening in the distal portion.

In accordance with another aspect of the present invention, an apparatus for closing an opening in a spinal disc is provided. The apparatus generally includes an elongate member including proximal and distal portions defining a longitudinal axis therebetween, and a closure device, such as those described above. The apparatus may include a constraint for releasably securing the closure device to the elongate member and/or securing the tissue engaging elements in the contracted condition, e.g., for facilitating delivery of the closure device into an opening in a spinal disc.

In one embodiment, the constraint may be a sheath overlying the elongate member. The sheath may include a lumen extending between its proximal and distal ends, and the closure device may be disposed within the lumen proximate the distal end of the sheath. The closure device may be inserted into the lumen such that the sheath may constrain the tissue engaging elements in the contracted condition. The elongate member may be a pusher member slidably disposed within the sheath, the pusher member including a substantially blunt distal end disposed proximate the distal end of the sheath.

In another embodiment, the elongate member may be a catheter or other tubular member, a rod, or a guidewire or other rail. The closure device may include a passage extending distally into a proximal end of the body for accommodating the distal portion of the elongate member therein. The passage may extend only partially into the body, the body having a substantially closed distal end. Alternatively, the passage may extend entirely through the body, and the closure device may include a plurality of leaflets, similar to the embodiment described above. The leaflets may be biased from an open condition for facilitating loading over the distal portion of the elongate member towards a closed condition for substantially closing the passage.

In accordance with yet another aspect of the present invention, a method is provided for closing an opening extending through the annulus fibrosis into an interior of a spinal disc. An opening may be created through the annulus fibrosis into the interior of the disc. A procedure may be performed within the interior of the disc through the opening, which may involve removing at least a portion of the nucleus pulposus, introducing a therapeutic agent into the interior, and/or implanting a device into the interior.

A closure device may be introduced into the opening, such as one of the closure devices described above. Generally, the closure device may include a body, and a plurality of tissue engaging elements extending from the body. The tissue engaging elements may be expandable from a contracted condition for facilitating introduction of the closure device into the opening to an expanded condition extending outwardly substantially transversely from a longitudinal axis of the closure device. The tissue engaging elements may be expanded to the expanded condition to substantially engage tissue surrounding the opening, thereby substantially securing the closure device within the opening.

The closure device may be carried by a distal portion of a delivery apparatus, such as that described above. To introduce the closure device into the opening, the distal portion of the delivery apparatus, with the closure device carried thereby, may be advanced into the opening. A sheath or other constraint may be removed, thereby releasing the closure device within the opening and/or releasing the tissue engaging elements, thereby allowing the tissue engaging elements to automatically expand to the expanded condition. In addition, to further engage the tissue engaging elements with tissue surrounding the opening, the closure device may be directed proximally within the opening, thereby substantially embedding the tissue engaging elements into the tissue surrounding the opening.

Alternatively, the closure device may be a threaded plug that may be threaded into the opening. Preferably, the threaded plug is a bioabsorbable body including a helical thread pattern along its outer surface. The plug may be detachably carried on the distal end of an elongate member. For example, the elongate member may have one or more cooperating elements for securing the plug to the distal end. With the plug directed into the opening, the elongate member may be twisted, threaded engaging the threads with the tissue surrounding the opening as the plug is threaded into the opening, thereby substantially sealing the opening. The plug may then be released from the distal end, and the elongate member removed.

Other objects and features of the present invention will become apparent from consideration of the following description taken in conjunction with the accompanying drawings.

### BRIEF DESCRIPTION OF THE DRAWINGS

FIGS. 1A and 1B are side views of a first preferred embodiment of a closure device, shown in its post-delivery and pre-delivery states, respectfully, in accordance with the present invention.

FIGS. 1C and 1D are proximal and distal end views of the closure device of FIG. 1A.

FIG. 2 is a cross-sectional side view of a distal portion of an apparatus for delivering the closure device of FIGS. 1A-1D, in accordance with the present invention.

FIGS. 3A-3C are cross-sectional views of a spinal disc, showing a method for sealing an opening in the spinal disc, in accordance with the present invention.

FIG. 4A is a front view of a second preferred embodiment of a closure device, in accordance with the present invention.

FIGS. 4B and 4C are cross-sectional views of the closure device of FIG. 4A, taken along line B-B.

FIG. 4D is a perspective side view of the closure device of FIG. 4A, with tissue engaging elements in a contracted condition.

FIGS. 5A and 5B are cross-sectional details of a tissue engaging element in expanded and contracted conditions, respectively.

FIG. 6 is a is a cross-sectional side view of a distal portion of an apparatus for delivering the closure device of FIGS. 4A-4D, in accordance with the present invention

FIGS. 7A and 7B are cross-sectional views of a spinal disc having an opening being closed using the apparatus of FIG. 6.

FIGS. 8A and 8B are side views of a third preferred embodiment of a closure device, in its post and pre-delivery states, respectfully, in accordance with the present invention.

FIG. 9 is a cross-sectional side view of a distal portion of an apparatus for delivering the closure device of FIGS. 8A-8B.

FIGS. 10A and 10B are cross-sectional views of a spinal disc, showing a method for sealing an opening in the spinal disc, in accordance with the present invention.

FIG. 11 is a side view of a fourth preferred embodiment of a closure device and an apparatus for delivering the closure device to close an opening in a spinal disc.

### DETAILED DESCRIPTION OF THE PREFERRED EMBODIMENTS

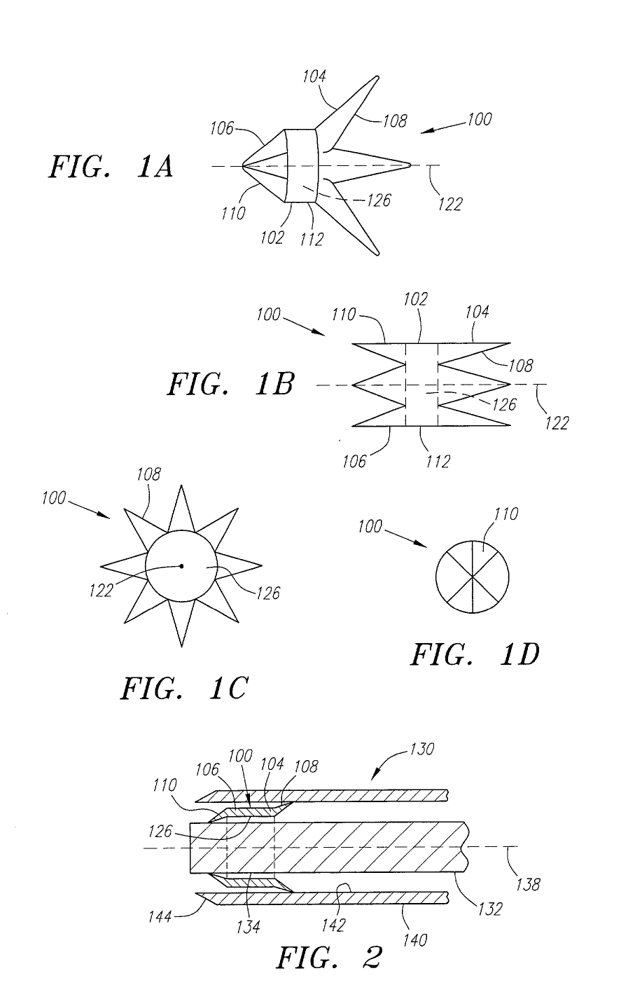

Turning now to the drawings, FIGS. 1A-1D show a first preferred embodiment of a closure device 100 for closing an incision, bore hole, or other opening communicating with an interior of a spinal disc (not shown). The closure device 100 is a generally annular-shaped member 102, including a proximal portion 104, a distal portion 106, and a passage 126 therethrough that extends substantially parallel to a longitudinal axis 122.

The proximal portion 104 includes a plurality of tissue engaging elements 108. Preferably, each tissue engaging element 108 is a tine terminating in a barb (not shown) configured for penetrating or otherwise engaging tissue. In a preferred embodiment, the tissue engaging elements 108 are disposed substantially symmetrically about the longitudinal axis 122 and/or in opposing pairs. The tissue engaging elements 108 are expandable from a contracted condition, preferably a substantially axial configuration, as shown in FIG. 1B, to an expanded condition, extending outwardly substantially transversely to the longitudinal axis 122, as shown in FIG. 1A.

The distal portion 106 includes a plurality of leaflets 110 that may be disposed substantially symmetrically about the longitudinal axis 122 and/or in opposing pairs. The leaflets 110 are expandable from a closed condition, as shown in FIG. 1A, for substantially closing the passage 126, towards an open condition, as shown in FIG. 1B. Alternatively, the leaflets 110 may be eliminated and the distal portion of the closure device 100 may be substantially closed (not shown). The proximal and distal portions 104, 106 may be connected to one another by a hinged region (not shown)about which the tissue engaging elements 108 and the leaflets 110 may pivot with respect to one another. Alternatively, the closure device 100 may include an intermediate tubular portion 112 from which the tissue engaging elements 108 and the leaflets 110 extend.

In a preferred embodiment, the annular-shaped member 102, the tissue engaging elements 108, and the leaflets 110 are integrally formed as a single tubular body, e.g., from a single sheet of material that is rolled, extruded, or otherwise formed into a tubular body. Portions of the tubular body may be removed using conventional methods, such as laser cutting, chemical etching, and the like, to form the tissue engaging elements 108 and/or the leaflets 110.

Preferably, the material is a superelastic alloy, such as a nickel-titanium (“Nitinol”) alloy. The tissue engaging elements 108 and leaflets 110 may be deflected to their expanded and closed conditions, respectively, as shown in FIG. 1A, and the material heat treated to set this shape into the material, as is well known to those skilled in the art. Thus, the tissue engaging elements 108 and the leaflets 110 may be biased towards the expanded and closed conditions of FIG. 1A, but may be temporarily constrained in the contracted and open conditions of FIG. 1B, as described further below. Alternatively, the tissue engaging elements 108 may be selectively directed between the contracted and expanded conditions, e.g., by plastically deforming the tissue engaging elements 108 and/or the leaflets 110 may selectively directed between the open and closed conditions, e.g., by plastically deforming the leaflets 110. In other alternatives, the tissue engaging elements 108 and/or leaflets 110 may be formed separately and attached to the annular-shaped member 102, for example, by welding or hinges.

As will be appreciated by those skilled in the art, a closure device in accordance with the present invention may have a variety of configurations, including two, three, four, five, or more tissue engaging elements, and/or including three or more leaflets (or optionally no leaflets if the distal portion is closed).

Turning to FIG. 2, an apparatus 130 is shown that may be used to deliver a closure device 100, such as that described above. Generally, the apparatus 130 includes an elongate member 132 including a proximal portion (not shown) and a distal portion 134, and defining a longitudinal axis 138. The elongate member 132 may be substantially rigid, semi-rigid, or substantially flexible, and may be formed from biocompatible materials, e.g., a metal, such as stainless steel, or a plastic. In one embodiment, the elongate member 132 may be a tubular catheter defining a lumen (not shown) for facilitating advancement of the elongate member 132 over a guidewire or other rail (not shown). Alternatively, the elongate member 132 may be a substantially solid rod or guidewire. A radiopaque marker or other locator element (not shown) may be provided on the distal portion 134 in a predetermined location relative to the closure device 100.

The apparatus 130 also includes a substantially flexible or semi-rigid tubular sheath 140 defining a lumen 142 extending between its proximal end (not shown) and its distal end 144. The distal end 144 has a size and shape to facilitate insertion into an opening in a spinal disc, e.g., having a tapered tip for facilitating substantially a traumatic introduction through the opening. The lumen 142 has a size for accommodating insertion of one or more devices therethrough, such as the closure device 100, guidewire, and the like (not shown).

The sheath 140 may include a seal (not shown), such as a hemostatic valve, within the lumen 142 and/or at or near the proximal end that provides a fluid-tight seal, yet accommodates insertion of one or more devices into the lumen 142, such as the elongate member 132 and/or closure device 100. Optionally, the sheath 140 may include a side port (not shown) that may communicate with the lumen 142, for example, to allow the infusion of fluids through the lumen 142 into the interior of a spinal disc or other treatment site.

The apparatus 130 may be used to deliver the closure device 100 to close and/or seal an incision, bore-hole, passage, or other opening through tissue (not shown), and particularly an opening providing access into the interior of a spinal disc. Alternatively, the apparatus 130 may be used to deliver the closure device 100 to engage tissue in other procedures, e.g., to connect tissue segments together or otherwise to secure tissue structures engaged by the closure device 100 with respect to one another.

Generally, the closure device 100 is pre-loaded on the distal portion 134 of the elongate member 132 with the sheath 140 overlying the closure device 100. For example, the proximal portion 104 of the closure device 100 may be directed over the distal portion 134 of the elongate member 132 until the distal portion 134 extends through the passage 126, thereby deflecting the leaflets 110 to the open condition, preferably outwardly to a substantially axial orientation, as shown in FIG. 1B.

Alternatively, if the distal portion of the closure device 100 is closed, the distal portion 134 of the elongate member 132 may only be partially received within the passage 126. In a further alternative, the passage 126 may be eliminated, and the distal portion 134 of the elongate member 132 may only abut the closure device 100 (not shown).

The elongate member 132 may then be introduced into the lumen 142 of the sheath 140, thereby constraining the tissue engaging elements 108 in the contracted condition. For example, the distal portion 134 of the elongate member 132 may be directed into the proximal end of the sheath 140, thereby deflecting the tissue engaging elements 108 into a substantially axial orientation within the lumen 142 of the sheath 140. The elongate member 132 may be advanced distally until the closure device 100 is disposed within the distal portion 144 of the sheath 140.

Alternatively, the closure device 100 may be loaded directly into the distal portion 144 of the sheath 140 with the tissue engaging elements 108 disposed in the contracted condition. The elongate member 132 may then be inserted through the lumen 142 of the sheath and into the passage 126 in the closure device 100, which may force the leaflets 110 into the open condition. Because the tissue engaging elements 108 of the closure device 100 are preferably biased to the expanded condition, they may engage an inner wall of the sheath 134, thereby constraining them in the contracted condition.

Alternatively, other constraints may be provided instead of or in addition to the sheath 140. For example, one or more filaments may be wrapped around the tissue engaging elements 108 and/or the closure device 100 generally to constrain the tissue engaging elements 108 in the contracted condition and/or to releasably secure the closure device 100 to the distal portion 134 of the elongate member 132. A slidable capsule, collar, or rolling membrane (not shown), such as those disclosed in U.S. Pat. Nos. 6,059,813, 5,690,644, 5,445,646, and 5,693,083, the disclosures of which are expressly incorporated herein by reference, may be provided that may engage the closure device 100 generally and/or the tissue engaging elements 108 specifically.

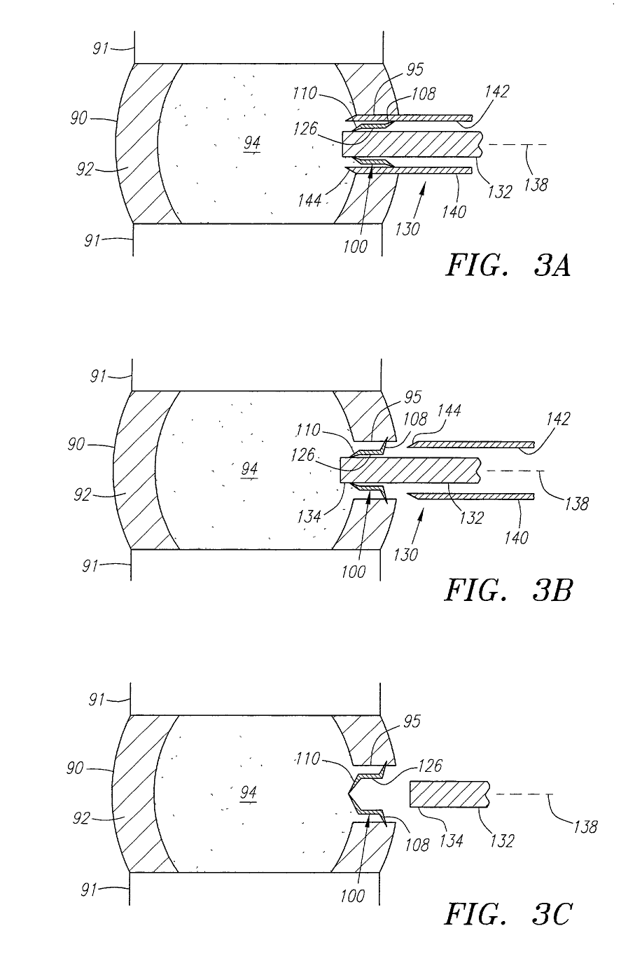

Turning to FIGS. 3A-3C, the apparatus 130 is shown being used to deliver the closure device 100 to substantially close an opening 95 in a spinal disc 90 between vertebrae 91. Details of the vertebrae 91 and disc 90 are omitted for clarity, but are well known to those skilled in the art. First, an opening 95 is created in the annulus fibrosis 92 of the disc 90 to gain access to the interior 94 of the disc 90. For example, the opening 95 may be a passage bored through the annulus fibrosis, a flap cut into the annulus fibrosis, and the like. A therapeutic and/or diagnostic procedure may be performed within the interior 94 of the disc 90. For example, at least a portion of the nucleus pulposus material may be removed from within the interior 94 of the disc 90, and an implant (not shown) may be introduced into the interior 94. Alternatively, a therapeutic agent may be introduced into the interior 94 via the opening 95. Additional information on procedures that may be performed within the disc are disclosed in co-pending application Serial No. ______, filed on the same day as the present application, entitled “Apparatus for Treating Spinal Disc” (attorney docket 257/045), the disclosure of which is expressly incorporated herein by reference.

First, with reference to FIG. 3A, upon completion of the procedure, the apparatus 130 may be introduced into the opening 95. If the apparatus 130 includes a locator element, e.g., a radiopaque marker, the apparatus 130 may be positioned using external imaging, e.g., fluoroscopy, x-ray, MRI, and the like, to dispose the closure device 100 within the opening 95. Alternatively, the opening 95 may be sufficiently large that direct visualization may be used. The apparatus 130 may be advanced over a guidewire or other rail (not shown) previously positioned through the opening 95 into the disc 90 or independently into the opening 95.

As shown in FIG. 3B, with the apparatus 130 properly positioned, the sheath 140 may be withdrawn to expose the closure device 100 within the opening 95. As the tissue engaging elements 108 are exposed, they may automatically expand substantially radially outwardly towards the expanded condition, thereby allowing them to substantially engage the annulus fibrosis tissue surrounding the opening 95. Preferably the tissue engaging elements 108 become substantially embedded within the annulus fibrosis tissue. Alternatively, the closure device 100 may be partially withdrawn from the interior 94 of the disc 90 to further enhance engagement with the surrounding annulus fibrosis tissue. The elongate member 132 may then be withdrawn from the opening 95, allowing the leaflets 110 to collapse to their closed condition, as shown in FIG. 3C.

Thus, a closure device in accordance with the present invention may be deployed within an opening in a spinal disc. The tissue engaging elements may substantially anchor the closure device within the opening, while the body and/or the tissue engaging elements effectively seal the opening. This may facilitate healing of the disc after a procedure and/or prevent nucleus pulposus or other materials within the disc from leaking, e.g., while the disc heals.

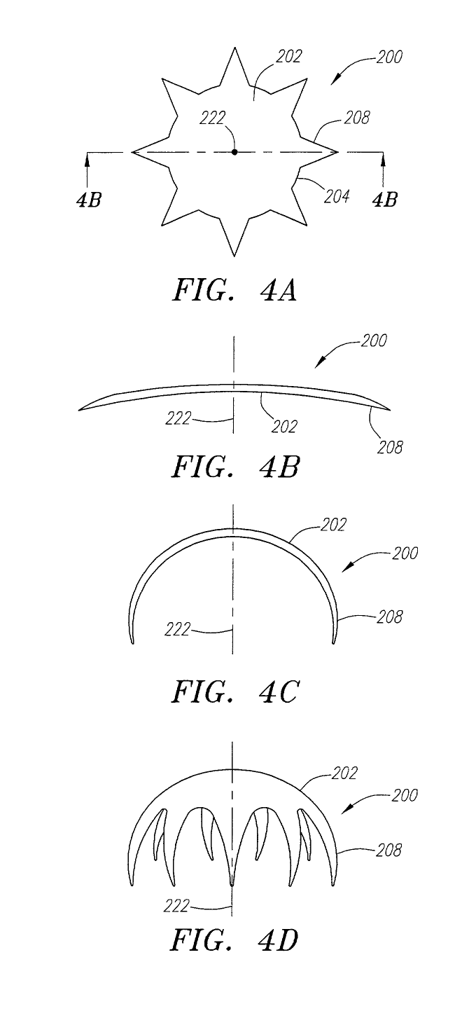

Turning to FIGS. 4A-4D, a second preferred embodiment of a closure device 200 is shown for closing an opening created through tissue, e.g., through an annulus fibrosis of a spinal disc (not shown). The closure device 200 includes a generally planar body 202 including an outer edge 204. An axis 222 extends substantially normal to a plane generally defined by the body 202, e.g., at a point of radial symmetry of the body 202. The body 202 may be truly planar or may have a slight concave shape in a relaxed state, i.e., when free from external forces, as best seen in FIGS. 4A and 4B. In addition, the body 202 may include a hole (not shown) therethrough, for example, along the axis 222, which may facilitate delivery of the closure device 200 over a guidewire or other rail (not shown).

A plurality of tissue engaging elements 208 extend from the outer edge 204 substantially transversely to the axis 222. The tissue engaging elements 208 are preferably tines terminating in pointed tips, which may include one or more barbs (not shown) configured for penetrating or otherwise engaging tissue. The tissue engaging elements 208 may be disposed substantially symmetrically about the outer edge 204 and/or in opposing pairs. As will be appreciated by those skilled in the art, the closure device 200 may include any desired number of tissue engaging elements 208, for example, two, three, four, or more.

In a preferred embodiment, the tissue engaging elements 208 are biased towards an expanded condition such that the tissue engaging elements 208 extend substantially tangentially from the body 202, as shown in FIGS. 4A and 4B. For example, the tissue engaging elements 208 may extend substantially perpendicular to the axis 222 if the body 202 is truly planar, or at an acute angle close to ninety degrees with respect to the axis 222 if the body 202 is slightly concave.

The tissue engaging elements 208 may be resiliently deflectable from the expanded condition to a contracted condition such that the tissue engaging elements 208 define a more acute angle with respect to the axis 222 than in the expanded condition, as shown in FIGS. 4C and 4D. For example, the tissue engaging elements 208 may be deflectable until they extend substantially parallel to the axis 222. As the tissue engaging elements 208 are deflected towards the contracted condition, the body 202 may become increasingly concave or may remain in substantially the same shape as when the tissue engaging elements 208 are in their expanded condition.

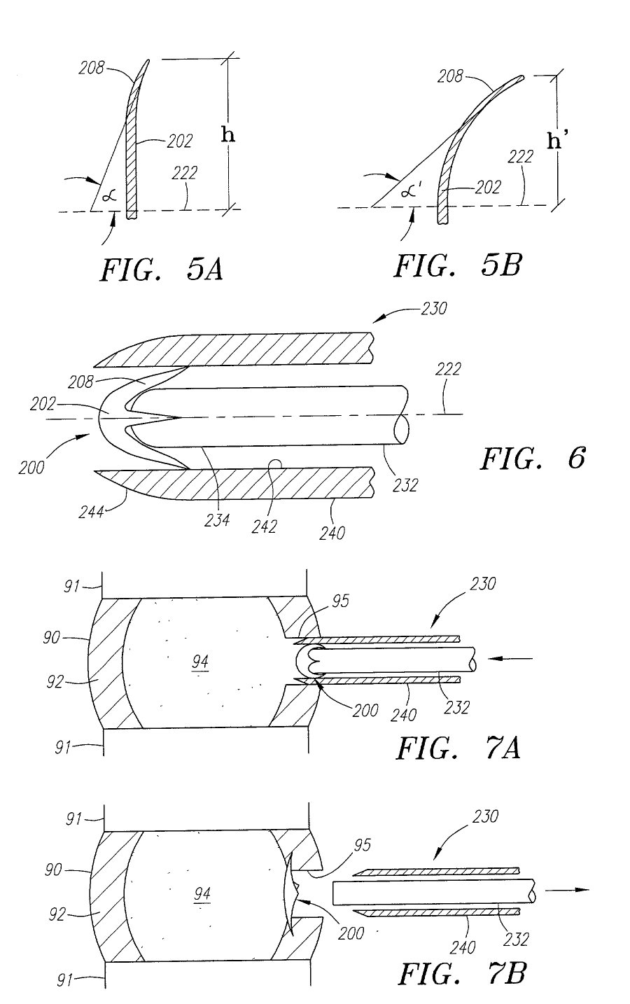

Turning to FIGS. 5A and 5B, a cross-section of an exemplary tissue engaging element 208 is shown. In FIG. 5A, the tissue engaging element 208 is shown in the expanded condition, defining an angle “α” with the axis 222. The angle α may be about ninety degrees if the body 202 is substantially planar, or alternatively, the angle α may be less than ninety degrees if the body 202 is biased to a slightly concave shape. Thus, the tissue engaging element 208 may have a height “h” between the axis 222 and the tip of the tissue engaging element 208.

In FIG. SB, the tissue engaging element 208 is shown in the contracted condition, defining an angle “α′” with the axis 222. The angle α′ is substantially more acute than angle α such that the tissue engaging element 208 has a height “h′” that is substantially smaller than the height “h.” Thus, the tissue engaging elements 208 may define a smaller cross-section in the contracted condition than in the expanded condition, thereby facilitating introduction of the closure device 100 into an opening (not shown).

Returning to FIGS. 4A-4D, in a preferred embodiment, the body 202 and the tissue engaging elements 208 are integrally formed from a single sheet of material, preferably a superelastic alloy, such as a nickel-titanium (“Nitinol”) alloy. Portions of the sheet may be removed using conventional methods, such as laser cutting, mechanical cutting, chemical etching, and the like, to form the tissue engaging elements 208. The sheet may be programmed to assume the generally planar configuration, e.g., using known heat treatment methods. Alternatively, the closure device 200 may be formed from a plastically deformable material, such as stainless steel.

Turning to FIG. 6, an apparatus 230 is shown that includes a closure device 200, such as that just described, an elongate member 232, and a sheath 240, similar to the embodiment described above. The elongate member 232 may be a substantially rigid or semi-rigid rod having a proximal end (not shown) and a distal end 234. Alternatively, the elongate member 232 may be a tubular member, such as a catheter, having a lumen (not shown) for receiving a guidewire or other rail therethrough. In this latter embodiment, the body 202 of the closure device 200 also includes a hole (not shown) that may be aligned with the lumen to receive a guidewire therethrough.

The sheath 240 may be a substantially flexible or semi-rigid tubular body including a lumen 242 extending between its proximal end (not shown) and distal end 244. The distal end 244 has a size and shape to facilitate insertion into an opening through tissue, such as the annulus fibrosis, e.g., having a tapered tip for facilitating substantially atraumatic introduction through the opening. The lumen 242 has a size for accommodating insertion of one or more devices therethrough, such as the closure device 200, the elongate member 232, a guidewire, and the like (not shown).

To prepare the apparatus 230 for use, the closure device 202 may be inserted into the lumen 242 at the proximal end of the sheath 240, thereby deflecting the tissue engaging elements 208 towards the contracted condition. The elongate member 232 may then be inserted into the lumen 242 at the proximal end of the sheath 240, and advanced distally, thereby directing the closure device 202 forward until it is disposed proximate the distal end 244 of the sheath 240.

Alternatively, the tissue engaging elements 208 may be directed to the contracted condition, and the closure device 200 inserted directly into the lumen 242, with the tissue engaging elements 208 oriented towards the proximal end of the sheath 240, as shown in FIG. 6. Preferably, if the body 202 of the closure device 200 is biased towards a slightly concave shape, the concave side of the body 202 is arranged towards the proximal end of the sheath 240, and the convex side of the body 202 is arranged towards the distal end 244 of the sheath 240.

Turning to FIGS. 7A and 7B, a method for delivering a closure device 200 using an apparatus 230 to substantially close and/or seal an opening 95 in a spinal disc 90. First, an opening 95 may be created, and a therapeutic and/or diagnostic procedure may be performed within the interior 94 of the disc 90. The closure device 200 may be carried by the distal end 234 of an elongate member 232, such as within the distal end 244 of the sheath 240, as described above. As shown in FIG. 7A, the distal end 244 of the sheath 240 may be inserted or otherwise positioned within the annulus fibrosis 92, i.e., through the opening 95, similar to the embodiments described above.

With the closure device 200 properly positioned (e.g., using direct or external visualization), the sheath 240 may be retracted, while the elongate member 232 holds the closure device 200 substantially stationary within the opening 95. As the sheath 240 is withdrawn, the tissue engaging elements 208 may be released and expanded to substantially engage the annulus fibrosis tissue surrounding the opening 95. Preferably, the tissue engaging elements 208 automatically expand towards the expanded condition, as described above, thereby substantially embedding the tissue engaging elements 208 within the tissue. The elongate member 232 may then be withdrawn, as shown in FIG. 7B, leaving the closure device 200 within the opening 95.

Preferably, the closure device 200 is arranged such that a concave side of the body 202 is oriented towards the exterior of the disc 90, while a convex side of the body 202 is oriented towards the interior 94 of the disc 90. Thus, after the closure device 200 has been deployed, relatively high pressure within the interior 94 of the disc 90 may be applied against the convex side of the body 202. This may force the body 202 to try to assume a more planar configuration, thereby further driving the tissue engaging elements 208 into the tissue surrounding the opening 95. Thus this embodiment may provide enhanced anchoring when subjected to increased internal pressure within the disc 90, as may occur during normal physical activity.

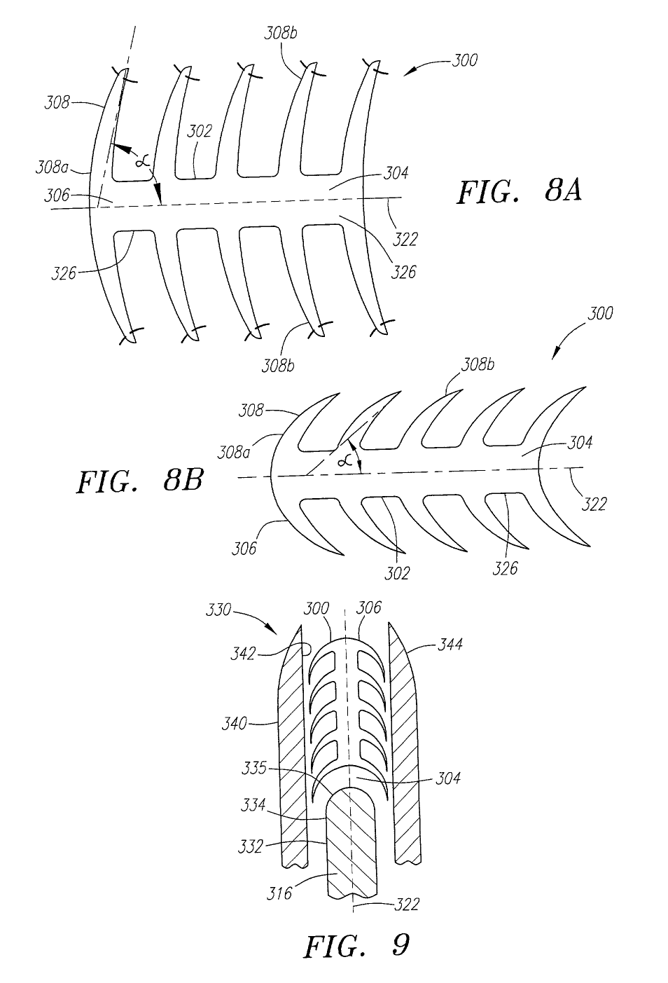

Turning to FIGS. 8A-8B, a third preferred embodiment of a closure device 300 is shown for closing an opening through tissue. The closure device 300 includes a generally elongate body 302, including a proximal end 304, a distal end 306, and a longitudinal axis 322 extending therebetween. The body 302 defines a peripheral surface 326 that extends between the proximal and distal ends 304, 306, and preferably defines a cylindrical surface that may have a substantially uniform diameter. Alternatively, the body 302 may be tapered in a predetermined manner, e.g., in a frustoconical shape or it may be tapered in an intermediate region and may be larger at the ends. A plurality of tissue engaging elements 308 extend along the peripheral surface 326 substantially perpendicularly to the longitudinal axis 322.

In a preferred embodiment, the tissue engaging elements 308 are a plurality of substantially annular flanges disposed adjacent one another along the longitudinal axis 322, e.g., between the proximal and distal ends 304, 306. Alternatively, the tissue engaging elements 308 may define portions of an annular flange, e.g., disposed adjacent one another about the peripheral surface 326 and/or along the longitudinal axis 322. In a further alternative, the tissue engaging elements 308 may be a helical thread (not shown) that extends along the peripheral surface 326, similar to the embodiment described further below.

Preferably, base portions 308a of the tissue engaging elements 308 are substantially fixed to the elongate body 302, while tips 308b of the tissue engaging elements 308 are deflectable towards one end (e.g., the proximal end 304) of the elongate body 302. Thus, the tissue engaging elements 308 may bend between an expanded condition for engaging surrounding tissue and a contracted condition for facilitating delivery, as described further below.

In the expanded condition, the tips 308b extend outwardly from the peripheral surface 326 substantially transversely to the longitudinal axis 322, as shown in FIG. 8A. In the expanded condition, the tissue engaging elements 308 may define an acute angle “α” with respect to the peripheral surface 326 along the longitudinal axis 322, preferably close to but less than ninety degrees. Thus, in a relaxed state, the tissue engaging elements 308 may be tilted slightly towards one end (e.g., the proximal end 304) of the elongate body 302.

In the contracted condition, the tips 308b may be bent towards one of the ends of the elongate body 302, preferably the proximal end 304, and at least partially towards the peripheral surface 326 of the elongate body 302, thereby reducing a cross-sectional profile of the closure device 300. In the contracted condition, the tips 108b may be oriented in a substantially axial configuration extending substantially parallel to the longitudinal axis 322, as shown in FIG. 8B.

The closure device 300 is generally formed from a biocompatible material, and/or from a bioabsorbable material. In one embodiment, the closure device 300 may be formed from a superelastic alloy, e.g., Nitinol, that may provide a resilient bias to the tissue engaging elements 308. Alternatively, the closure device 300 may be formed from a bioabsorbable material, e.g., a naturally occurring extra-cellular matrix material, such as intestinal submucosa, stomach submucosa, bladder submucosa, and the like.

Turning to FIGS. 9, an apparatus 330 is shown that may be used to deliver a closure device 300, such as that described above. Generally, the apparatus 330 includes an elongate member 332 including a proximal portion (not shown), a distal portion 334, terminating in a substantially blunt distal end 335 and a longitudinal axis 322. The elongate member 332 may be substantially rigid, semi-rigid, or substantially flexible, and may be formed from biocompatible materials, such as stainless steel. Preferably, the elongate member 332 is a tubular member defining a lumen 316 for facilitating advancement of the elongate member 332 over a guidewire or other device (not shown). Alternatively, the elongate member 332 may be a guidewire, a solid bumper device, and the like.

A radiopaque marker or other positioning element (not shown) may be provided on the distal portion 334. Alternatively, a marker may be provided on the sheath and/or the closure device.

The apparatus 330 also includes a substantially flexible or semi-rigid tubular sheath 340 defining a lumen 342 extending between its proximal end (not shown) and its distal end 344. Similar to the embodiments described above, the distal end 344 has a size and shape to facilitate insertion into an opening in a spinal disc and/or the lumen 342 may have a size for accommodating insertion of one or more devices therethrough.

The sheath 340 may include a seal (not shown), such as a hemostatic valve, within the lumen 342 and/or at or near the proximal end that provides a fluid-tight seal, yet accommodates insertion of one or more devices into the lumen 342, such as the elongate member 332 and/or closure device 300. Optionally, the sheath 340 may include a side port (not shown) that communicates with the lumen 342, for example, to allow the infusion of fluids through the lumen 342 into the interior of a spinal disc or other treatment site.

The apparatus 330 may be used to deliver the closure device 300 to close and/or seal an incision, bore-hole, passage, or other opening through tissue (not shown), and particularly an opening providing access into the interior of a spinal disc. Alternatively, the apparatus 330 may be used to deliver the closure device 300 to engage tissue in other procedures, e.g., to connect tissue segments together or otherwise to secure tissue structures engaged by the closure device 300 with respect to one another. Generally, the closure device 300 is pre-loaded within the lumen 342 of the sheath 340 proximate the distal end 344, similar to the embodiments described above.

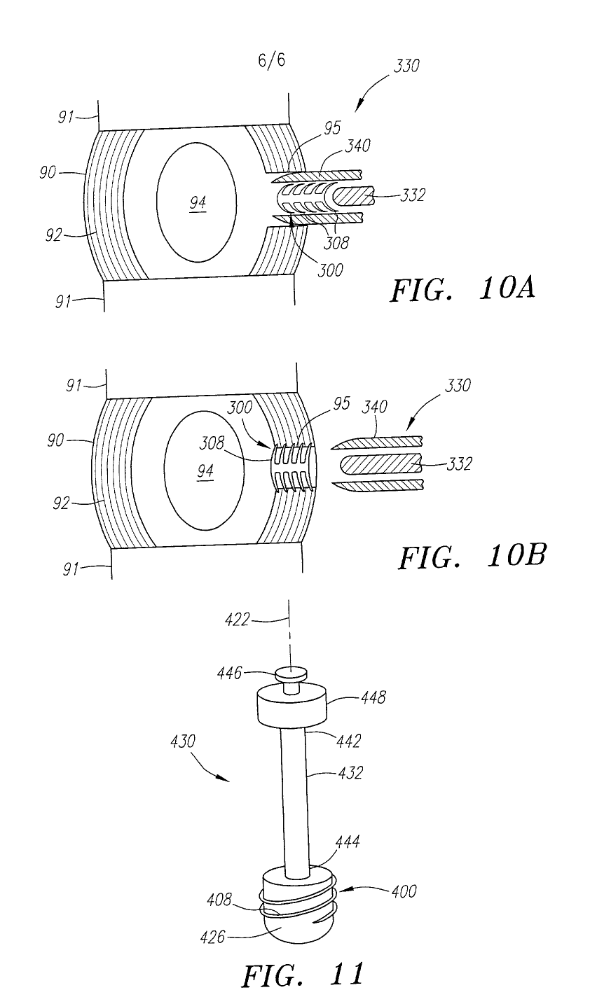

Turning to FIGS. 10A and 10B, the apparatus 330 may be used to deliver the closure device 300 to substantially close and/or seal an opening in a spinal disc 90. First, an opening 95 is created in the annulus fibrosis 92 of the disc 90 to gain access to the interior 94 of the disc 90. For example, the opening 95 may be a passage bored through the annulus fibrosis, a flap cut into the annulus fibrosis, and the like. A therapeutic and/or diagnostic procedure may be performed within the interior 94 of the disc 90, as described above.

Upon completion of the procedure, as shown in FIG. 10A, the apparatus 330 may be introduced into the opening 95. Once the apparatus 330 is properly positioned, the sheath 340 may be withdrawn to expose the closure device 300 within the opening 95, as shown in FIG. 10B. The elongate member 332 generally abuts the closure device keeping the closure device 300 in place. As the tissue engaging elements 308 are exposed, the tips may automatically expand radially outward towards the expanded condition, thereby causing the tissue engaging elements 308 to substantially engage the annulus fibrosis tissue surrounding the opening 95, as shown in FIG. 10B.

Preferably, the tissue engaging elements 308 become substantially embedded within the annulus fibrosis tissue. Alternatively, the closure device 300 may be partially withdrawn from the interior 94 of the disc 90 to further enhance engagement with the surrounding annulus fibrosis tissue. The elongate member 332 may then be withdrawn from the opening 95, allowing the opening 95 to close.

Turning to FIG. 11, yet another apparatus 430 is shown for sealing or otherwise closing an opening through tissue, such as the annulus fibrosis of a spinal disc (not shown). Generally, the apparatus 430 includes a threaded plug 400 that is releasably secured to the distal end 444 of an elongate member 432. Preferably, the threaded plug 400 is a bioabsorbable body including one or more helical threads 408 extending along its peripheral surface 426. The plug 400 and the distal end 444 of the elongate member 432 may include cooperating elements for releasably securing the plug 400 on the distal end 444, such as cooperating threads, gripping elements and pockets, and the like (not shown). For example, co-pending application Ser. No. 09/738,431, filed Dec. 12, 2000, discloses a bioabsorbable plug and delivery apparatus that may be appropriate for use with the present invention. The disclosure of this application and any others cited therein are expressly incorporated herein by reference. The proximal end 442 of the elongate member 432 may include an actuator 446 on a handle 448 for releasing the cooperating elements.

Similar to the embodiments described above, the threaded plug 400 that may be advanced into an opening in a spinal disc (not shown) such that the thread(s) 408 substantially seal the opening. For example, the plug 400 may be used to close an opening after a procedure, such as one or more of those procedures disclosed in the application incorporated by reference above. Preferably, with the plug 400 directed into the opening, the elongate member 432 may be twisted, thereby threading the plug 400 into the opening. As the plug 400 is threaded, the thread(s) 408 may substantially engage the tissue surrounding the opening (either directly or indirectly, e.g., by engaging liner material extending from the interior through the opening). The plug 400 may then be deployed, e.g., by activating the actuator 446 to release the plug 400 from the distal end of the elongate member 432. The elongate member 432 may then be removed from the patient's body.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1. A device for closing an opening in a spinal disc, comprising:
a body defining a longitudinal axis; and
a plurality of tissue engaging elements on the body, the tissue engaging elements being expandable from a contracted condition towards an expanded condition, the tissue engaging elements extending outwardly from the body substantially transversely to the longitudinal axis in the expanded condition.

2. The device of claim 1, wherein the body comprises a generally annular-shaped member defining a passage extending therethrough substantially parallel to the axis, and wherein the device further comprises a plurality of leaflets on the annular-shaped member, the leaflets being biased from an open condition towards a closed condition, the leaflets being oriented towards one another in the closed condition for substantially closing an opening in the distal portion.

3. The device of claim 2, wherein the leaflets extend from a distal portion of the body portion, the leaflets being oriented in a substantially axial configuration in the open condition for accommodating loading the annular-shaped member over a delivery device.

4. The device of claim 2, wherein the leaflets are disposed symmetrically about the longitudinal axis.

5. The device of claim 1, wherein the body comprises a superelastic alloy.

6. The device of claim 1, wherein the body comprises Nitinol.

7. The device of claim 1, wherein the tissue engaging elements extend from a proximal portion of the body, and wherein the tissue engaging elements are oriented in a substantially axial configuration in the contracted condition.

8. The device of claim 1, wherein the tissue engaging elements are disposed substantially symmetrically about the longitudinal axis.

9. The device of claim 1, wherein the tissue engaging elements comprise barbs for penetrating tissue.

10. The device of claim 1, wherein the tissue engaging elements are biased towards the expanded condition.

11. The device of claim 1, wherein the body comprises a generally planar body comprising an outer edge, the axis being substantially normal to a surface of the planar body, the tissue engaging elements extending from the outer edge of the planar body.

12. The device of claim 11, wherein the planar body defines a concave shape in a relaxed state free from external forces.

13. The device of claim 11, wherein the tissue engaging elements are deflected towards an axial orientation substantially parallel to the longitudinal axis in the contracted condition.

14. The device of claim 13, wherein the planar body is deflectable to assume a concave shape when the tissue engaging elements are deflected towards the contracted condition.

15. The device of claim 11, wherein the tissue engaging elements are biased towards a generally planar configuration lying within a plane defined by the planar body.

16. The device of claim 1, wherein:
the body comprises an elongate body defining a peripheral surface extending between proximal and distal ends of the elongate body, the elongate body comprising a biocompatible material; and
the tissue engaging elements extend along the peripheral surface substantially perpendicular to the longitudinal axis, tips of the tissue engaging elements being deflectable towards the elongate body to define the contracted condition, the tips being biased towards the expanded condition wherein the tips extend substantially transversely from the peripheral surface.

17. The device of claim 16, wherein, in a relaxed state, the tissue engaging elements define an acute angle with the peripheral surface along the longitudinal axis.

18. The device of claim 16, wherein the tissue engaging elements comprise a plurality of tissue engaging elements disposed adjacent to one another along the longitudinal axis.

19. The device of claim 18, wherein the tissue engaging elements on the body are disposed substantially symmetrically about the longitudinal axis.

20. The device of claim 18, wherein the tissue engaging elements comprise a plurality of annular flanges extending from the peripheral surface.

21. The device of claim 20, wherein the annular flanges are disposed adjacent one another along the longitudinal axis.

22. The device of claim 18, wherein the tissue engaging elements comprise one or more helical threads extending along the peripheral surface.

23. A device for closing an opening in a spinal disc, comprising:
an elongate body comprising proximal and distal ends defining a longitudinal axis therebetween, and a peripheral surface extending between the proximal and distal ends, the elongate body comprising a biocompatible material;
one or more tissue engaging elements extending along the peripheral surface substantially perpendicular to the longitudinal axis, tips of the tissue engaging elements being deflectable towards a contracted condition wherein the tips are directed towards the elongate body to facilitate insertion into an opening in a spinal disc, the tips being biased towards an expanded condition wherein the tips extend substantially transversely from the peripheral surface.

24. A device for closing an opening in a spinal disc, comprising:
a generally annular-shaped member defining a longitudinal axis extending between proximal and distal portions;
a plurality of tissue engaging elements on the proximal portion, the tissue engaging elements being biased from a contracted condition towards an expanded condition, the tissue engaging elements extending outwardly from the body portion substantially transversely to the longitudinal axis in the expanded condition; and
a plurality of leaflets on the distal portion, the leaflets being biased from an open condition towards a closed condition, the leaflets being oriented towards one another in the closed condition for substantially closing an opening in the distal portion.

25. A device for closing an opening in a spinal disc, comprising:
a generally planar body comprising an outer edge, the planar body defining a longitudinal axis substantially normal to a surface of the planar body, the planar body being biased towards a concave shape; and
a plurality of tissue engaging elements extending from the outer edge of the planar body, the tissue engaging elements being biased from a contracted condition towards an expanded condition, the tissue engaging elements extending outwardly from the outer edge substantially transversely to the longitudinal axis in the expanded condition.

26. An apparatus for closing an opening in a spinal disc, comprising:
an elongate member comprising proximal and distal portions defining a longitudinal axis therebetween;
a closure device proximate the distal portion of the elongate member, the closure device comprising a body and a plurality of tissue engaging elements on the body, the tissue engaging elements being biased to expand from a contracted condition to an expanded condition wherein the tissue engaging elements extend outwardly substantially transversely to the longitudinal axis; and
a constraint for releasably securing the tissue engaging elements in the contracted condition for facilitating delivery of the closure device into an opening in a spinal disc.

27. The apparatus of claim 26, wherein the constraint comprises a sheath overlying the elongate member.

28. The apparatus of claim 27, wherein the sheath comprises a lumen extending between its proximal and distal ends, and wherein the closure device is disposed within the lumen proximate the distal end of the sheath, the sheath constraining the tissue engaging elements in the contracted condition.

29. The apparatus of claim 28, wherein the elongate member comprises a pusher member slidably disposed within the sheath, the pusher member comprising a substantially blunt distal end disposed proximate the distal end of the sheath.

30. The apparatus of claim 28, further comprising a locator element on the sheath proximate the distal end of the sheath for facilitating positioning the closure element within an opening in a spinal disc.

31. The apparatus of claim 28, wherein the body comprises a generally planar body comprising an outer edge, and wherein the tissue engaging elements extending from the outer edge of the planar body.

32. The apparatus of claim 31, wherein the tissue engaging elements are deflected towards a generally axial orientation substantially parallel to the longitudinal axis within the lumen of the sheath to define the contracted condition.

33. The apparatus of claim 32, wherein the planar body is deflectable to assume a concave shape when the tissue engaging elements are deflected towards the contracted condition.

34. The apparatus of claim 31, wherein the body and the tissue engaging elements are biased towards a generally planar configuration.

35. The apparatus of claim 28, wherein the body comprises an elongate body defining a peripheral surface extending between its proximal and distal ends, and the tissue engaging elements extend along the peripheral surface substantially perpendicular to the longitudinal axis, tips of the tissue engaging elements being deflectable towards the elongate body to define the contracted condition, the tips being biased towards the expanded condition wherein the tips extend substantially transversely from the peripheral surface.

36. The apparatus of claim 35, wherein, in a relaxed state, the tissue engaging elements define an acute angle with the peripheral surface.

37. The apparatus of claim 35, wherein the tissue engaging elements comprise a plurality of annular flanges extending from the peripheral surface, the annular flanges being disposed adjacent one another along the longitudinal axis.

38. The apparatus of claim 26, wherein the elongate member comprises a tubular member.

39. The apparatus of claim 26, wherein the elongate member comprises a guidewire.

40. The apparatus of claim 26, further comprising a locator element on the distal portion of the elongate member for facilitating positioning the closure element within an opening in a spinal disc.

41. The apparatus of claim 26, wherein the closure device comprises a passage extending distally into a proximal end of the body for accommodating the distal portion of the elongate member therein.

42. The apparatus of claim 41, wherein the passage extends only partially into the body, the body having a substantially closed distal end.

43. The apparatus of claim 41, wherein the passage extends entirely through the body, and wherein the closure device further comprises a plurality of leaflets biased from an open condition for facilitating loading over the distal portion of the elongate member towards a closed condition for substantially closing the passage.

44. A method for closing an opening extending through annulus fibrosis into an interior in a spinal disc, the method comprising:
creating an opening through the annulus fibrosis into the interior of the disc;
performing a procedure within the interior of the disc;
introducing a closure device into the opening, the closure device comprising a body, and a plurality of tissue engaging elements extending from the body, the tissue engaging elements being expandable from a contracted condition for facilitating introduction of the closure device into the opening to an expanded condition extending outwardly substantially transversely from a longitudinal axis of the closure device; and
expanding the tissue engaging elements to the expanded condition to substantially engage tissue surrounding the opening, thereby substantially securing the closure device within the opening.

45. The method of claim 44, wherein the closure device is carried by a distal portion of a delivery apparatus, and wherein the step of introducing the closure device comprises introducing the distal portion of the delivery apparatus into the opening.

46. The method of claim 45, wherein the body comprises a generally annular shaped body defining a passage therethrough, and the delivery apparatus comprises an elongate member, and a distal portion of the elongate member is disposed through the passage such that the closure device is carried by the elongate member.

47. The method of claim 46, wherein the closure device further comprises a plurality of leaflets on the body, the leaflets being biased from an open condition towards a closed condition, the leaflets being oriented towards one another in the closed condition.

48. The method of claim 47, further comprising withdrawing the elongate member from within the passage to deploy the closure device within the opening, whereupon the leaflets automatically move towards the closed condition to substantially close the passage.

49. The method of claim 45, wherein the tissue engaging elements are biased to the expanded condition, and wherein the delivery apparatus comprises a constraint for releasably securing the tissue engaging elements in the contracted condition.

50. The method of claim 49, wherein the step of expanding the tissue engaging elements comprises removing the constraint from the tissue engaging elements.

51. The method of claim 49, wherein the constraint comprises a sheath disposed over the closure device.

52. The method of claim 51, wherein the step of expanding the tissue engaging elements comprises withdrawing the sheath from over the closure device.

53. The method of claim 52, wherein the step of expanding the tissue engaging elements further comprises directing the body proximally within the opening, thereby substantially embedding the tissue engaging elements into the tissue surrounding the opening.

54. The method of claim 51, wherein the body comprises an elongate body defining a peripheral surface, and wherein the tissue engaging elements extend along the peripheral surface substantially perpendicular to the longitudinal axis.

55. The method of claim 51, wherein the delivery apparatus comprises a pusher member slidably disposed within the sheath, the method further comprising withdrawing the sheath the deploy the closure device within the opening, the pusher member preventing substantial proximal movement of the closure device as the sheath is withdrawn.

56. The method of claim 55, wherein the body comprises a generally planar body defining an outer edge, and wherein the tissue engaging elements extend from the outer edge, and wherein the closure device is disposed within the sheath such that the tissue engaging elements are deflected towards a proximal end of the sheath.

57. The method of claim 56, wherein the planar body and the tissue engaging elements are biased towards a generally planar configuration when the sheath is withdrawn, the tissue engaging elements substantially engaging the tissue surrounding the opening in the generally planar configuration.

58. The method of claim 44, wherein the tissue engaging elements comprise a plurality of annular flanges extending about the peripheral surface, the annular flanges being disposed adjacent one another along the longitudinal axis.

59. The method of claim 58, wherein the annular flanges define an acute angle with the longitudinal axis towards a proximal end of the elongate body.

60. The method of claim 44, wherein the step of performing a procedure comprises removing at least a portion of the nucleus pulposus material from the interior of the spinal disc.

61. The method of claim 44, wherein the step of performing a procedure comprises introducing an implant within the interior of the spinal disc.

62. The method of claim 44, wherein the step of performing a procedure comprises introducing a therapeutic agent into the interior of the spinal disc.

63. The method of claim 44, wherein the closure device is disposed on a distal portion of a delivery apparatus, the distal portion comprising a locator element, and wherein the step of introducing of a closure device comprises positioning the closure device while monitoring the locator element with an external imaging device.

64. A method for closing an opening extending through annulus fibrosis into an interior in a spinal disc, the method comprising:
creating an opening through the annulus fibrosis into the interior of the disc;
performing a procedure within the interior of the disc;
threading a plug into the opening, the plug comprising one or more helical threads extending along a peripheral surface, the one or more helical threads substantially engaging a periphery surrounding the opening, thereby substantially sealing the opening.

65. The method of claim 64, wherein the plug is carried on a distal end of an elongate member, and wherein the threading step comprises twisting the elongate member to thread the plug into the opening.

66. The method of claim 65, further comprising releasing the plug from the elongate member.

67. The method of claim 64, wherein the plug comprises bioabsorbable material.

68. The method of claim 64, wherein the step of performing a procedure comprises removing at least a portion of the nucleus pulposus material from the interior of the spinal disc.

69. The method of claim 64, wherein the step of performing a procedure comprises introducing an implant within the interior of the spinal disc.

70. The method of claim 64, wherein the step of performing a procedure comprises introducing a therapeutic agent into the interior of the spinal disc.

71. The method of claim 64, wherein the one or more helical threads substantially engage tissue surrounding the opening.

72. The method of claim 64, wherein the step of performing a procedure comprises lining an interior of the disc with a liner material such that a portion of the liner material extends into the opening.

73. The method of claim 72, wherein the one or more helical threads substantially engage the portion of the liner material extending into the opening.
